(19) United States
(12) Patent Application Publication
(10) Pub. No.: US 2024/0335509 A1
(43) Pub. Date: Oct. 10, 2024

(54) TREATMENT OF BONE TISSUE TO INCREASE THE BIOAVAILABILITY OF GROWTH FACTORS

(71) Applicant: RED ROCK REGENERATION INC., Toronto (CA)

(72) Inventors: Sean A.F. Peel, Oakville (CA); Sowmya Shivanna, Toronto (CA)

(21) Appl. No.: 18/712,842

(22) PCT Filed: Nov. 25, 2022
(86) PCT No.: PCT/CA2022/051736

§ 371 Date: May 23, 2024

Related U.S. Application Data
(60) Provisional application No. 63/264,593, filed on Nov. 26, 2021.

(51) Int. Cl. A61K 38/18; A61K 35/32; A61P 19/08; C07K 14/51; C12N 5/077

(52) U.S. Cl. CPC A61K 38/18; A61K 35/32; A61P 19/08; C07K 14/51; C12N 5/0654; C12N 2500/14; C12N 2500/46

(57) ABSTRACT

A process is provided for treating demineralized bone (DB) comprising incubating the DB with a solution of a chaotropic agent at a temperature in the range of about 12° C. to about 30° C. to increase the release of a bone growth factor followed by selectively removing the chaotropic agent while retaining the released growth factor and the DB. A product prepared by the process is also provided as is the use of said product to promote bone formation or bone fusion in a subject in need thereof.

### CROSS REFERENCE

The present application claims the benefit of U.S. Provisional Application 63/264,593 filed Nov. 26, 2021.

### FIELD

The present description relates to processing of bone tissue and more particularly to processing demineralized bone (DB). The description further relates to the products of said process and to uses of said product.

### BACKGROUND

Bone matrix comprises a mineral phase and an organic phase. The organic phase contains proteins, proteoglycans, glycosaminoglycans and lipids. The structural protein collagen type I makes up approximately 90% of the organic phase by dry weight. The non-collagenous proteins include a number of growth factors. Growth factors in bone matrix include members of the bone morphogenetic protein (BMP) family, the transforming growth factor beta family (TGF-B), the insulin-like growth factor (IGF) family, the fibroblast growth factor family (FGF), the platelet derived growth factor (PDGF) family and the vascular endothelial growth factor (VEGF) family (Devescovi et al., Chir Organi Mov (2008) 92:161-168). The assignment of growth factors into these families is primarily based on structural similarities and the receptors they interact with.

Removal of the mineral phase from bone makes the demineralized bone matrix (DBM) osteoinductive (OI) (Urist M. R., Science (1965) 150:893-899) by making growth factors in the matrix bioavailable. Specifically, it is the BMPs, including BMP-2, -4, -6 and -7, that are the growth factors responsible for the osteoinductive activity of demineralized bone matrix (Sampath & Reddi, Proc. Natl Acad. Sci. USA (1981) 78:7599-7603; Wang et al., PNAS (1998) 85:9484-9488), by stimulating the differentiation of multipotent stromal cells (MSC) into osteoblasts. Non-BMP growth factors in bone matrix also stimulate bone repair; by promoting angiogenesis (VEGF, FGF-1 & 2, PDGF-BB), stimulating chemotaxis and proliferation of MSCs (PDGF-BB, TGF-ß1), and stimulating production of bone matrix by osteoblasts (IGF-1, IGF-2) (Devescovi et al., Chir Organi Mov (2008) 92:161-168).

This has led to the use of demineralized bone matrix (DBM) to promote bone repair clinically in dental, orthopedic and spine surgery (Gruskin et al., Advanced Drug Delivery Reviews (2012) 64: 1063-1077) where it is has been used either alone or in combination with a carrier such as poloxamer P407, glycerol, hyaluronic acid, or carboxymethyl cellulose. However, there is considerable lot-to-lot variability in OI and other biological activities and there remain questions as to the clinical effectiveness of DBM.

The osteoinductive activity of DBM has been correlated to the amount of various bone growth factors within the bone matrix, especially BMP-2 and BMP-7 (Murray et al., ATLA (2007) 35, 405-409). Pieterzak et al. demonstrated that the amount of BMP-2, 4 and 7 extracted from DBM using the chaotrope guanidine HCl is practically unchanged even after 7 days of incubation in buffer at 37° C. (Pieterzak et al., J. Craniofac. Surg. (2017) 28: 2183-8 2188), suggesting that BMPs and possibly the other non-BMP bone matrix growth factors remain locked away within the collagenous matrix even after demineralization and thus are not available to act during the early phases of bone repair minimizing their potential beneficial effects.

Studies have also demonstrated that variables in processing of demineralized bone matrix, including particle size, extent of demineralization and chemical treatment influence the osteoinductive activity, BMP content and BMP inhibitor content (Zhang et al. J. Periodont. (1997) 65:1085-1092; Pieterzak et al., Cell Tissue Bank (2011) 12:81-88; Benham et al., Connective Tissue Research (2004) 45: 257-260).

Various growth factors have been extracted from demineralized bone matrix using chaotropic agents such as urea or guanidine HCl including: BMP-2, BMP-4, BMP-6, BMP-7, TGF-ß1, PDGF-BB, FGF-1, FGF-2, IGF-1, IGF-2 (Wang et al., Proc. Nat. Acad. Sci. USA (1988) 85: 9484-9488; Sampath et al., J.Bio.Chem (1990) 265: 13198-13205; Seyedin et al., Proc. Nat. Acad. Sci. USA (1985) 82: 2267-2271; Hauschka et al., J. Bio. Chem. (1986) 261: 12665-12674, Mohan et al., Biochimica et Biophysica Acta (General) (1988) 966: 44-55; Mohan, Growth Factors (1990), 2:267-271). During the isolation of these growth factors, to ensure that they were not degraded but remained active, the incubation with chaotropes and the subsequent dialysis steps were performed at 2-8° C. in the presence of protease inhibitors.

Urist et al. demonstrated that if HCl demineralized bone was incubated with phosphate buffer prior to implantation there was a significant loss in osteoinductive activity (Urist et al., Proc. Nat. Acad. Sci. USA (1973) 70: 3511-3515) which they attributed to the degradation of the BMP by an endogenous enzyme.

Sampath & Reddi (Proc. Nat. Acad. Sci. USA (1981) 78: 7599-7603) extracted DBM with 4M Guanidine HCl, 50 mM Tris pH 7.0 with protease inhibitors at 4° C. for 16 hours and then transferred both the extract and the residue to dialysis tubing and dialyzed extensively against distilled water at 4° C. for 3-4 days and then co-lyophilized the matrix and extract and tested it for osteoinductive activity in vivo. They determined that there was no significant difference in the activity between the original DBM and the reconstituted matrix.

Therefore, while the prior art teaches that it is possible to extract growth factors from bone matrix and reconstitute them with the matrix it also teaches the necessity to perform the steps at 2 to 8° C. in the presence of protease inhibitors to prevent degradation of the growth factors by endogenous enzymes and to maintain the osteoinductive activity of the matrix, and further teaches that when this is done and the extracted growth factors are reconstituted with the residual bone matrix there is no demonstrated increase in osteoinductive activity.

Therefore, there is a need for improved processes for treating demineralized bone to increase bioavailability of bone growth factors and the biological activity of the matrix and for the products produced by such process.

### SUMMARY

Accordingly, in one aspect, there is provided a process for treating demineralized bone (DB) comprising:
incubating the DB with an aqueous solution of a chaotropic agent at a temperature in
the range of about 12° C. to about 30° C. to increase the release of a bone growth factor,
selectively removing the chaotropic agent while retaining the released growth factor with the DB.

In an embodiment, the aqueous solution of the chaotropic agent further includes a salt, and in particular an inorganic salt.

In yet a further embodiment, the process is carried out in the absence of an added protease inhibitor.

In another embodiment there is provided a process for treating demineralized bone (DB) comprising:
incubating the DB with an aqueous solution of a chaotropic agent at temperature in the range of about 2° C. to about 30° C. to increase the release of a bone growth factor,
selectively removing the chaotropic agent while retaining the released growth factor with the DB and
wherein the process is carried out the absence of added protease inhibitor.

In another aspect, there is provided a product produced by the process for treating demineralized bone as described above.

In another aspect there is provided a use of the product produced by the process, to promote bone formation and/or bone fusion.

In a further embodiment the use of the product produced by the process, is to promote bone formation in boney voids and bone defects.

In still a further embodiment the use of the product produced by the process is to promote bone fusion of two separate bones or of non-unions. In still a further aspect the two separate bones comprise vertebra or ankle bones and the non-union comprises fracture non-unions. In a further aspect the use comprises applying the product at the site where bone formation or bone fusion is to occur.

In another aspect there is provided a method of treating a patient in need thereof with the product of the process, to promote bone formation and/or bone fusion.

In a further embodiment, the method comprises administering the product into boney voids or bone defects.

In a further embodiment the method comprises promoting fusion of two separate bones or treating non-unions by administering the product at the site where fusion is to occur. In still a further embodiment the two separate bones comprise vertebra, or ankle bones and the non-union comprises fracture non-unions.

In another aspect there is provided a product for use in bone formation and/or bone fusion.

In a further embodiment the product is for use in bone formation in bone voids or bone defects.

In a further embodiment the bone fusion is fusion of two separate bones or of non-unions. In a further embodiment the two separate bones comprise vertebra or ankle bones and the non-union comprises fracture non-unions. In a further aspect the product for use is applied at the site where bone formation or bone fusion is to occur.

### BRIEF DESCRIPTION OF DRAWINGS

Embodiments will now be described with reference to the appended drawings wherein:

FIG. 1 is a diagram depicting a tangential flow filtration system for use to selectively remove the chaotrope from the solution after it has released bone growth factors from the demineralized bone.

FIG. 2 is a graph depicting the results of an in vitro osteoinductive (OI) assay measuring the alkaline phosphatase activity of C2C12 cells incubated with inactive DBM, DBM or demineralized bone treated with a chaotropic solution as described herein (NMP) all prepared from the same pool of bovine bone particulate.

### DETAILED DESCRIPTION

The term “demineralized bone matrix” will be understood to mean bone matrix that has been treated to reduce the amount of calcium present in the matrix. This can be done by treatment with a strong acid (e.g. HCl) or weak acid (e.g. acetic acid) or a chelating agent (e.g. EDTA)

The term “fully demineralized bone matrix” will be understood to mean bone matrix that has a calcium content of less than or equal to 8% w/w.

The term “partially demineralized bone matrix” will be understood to mean bone matrix that has a calcium content of greater than 8% w/w but less than the original calcium content.

The term “demineralized bone”, “DB”, as used herein refers to bones, bone segments, bone fragments, bone fibers or bone particulates the matrix of which has been fully or partially demineralized wherein the fully demineralized bone has a calcium content of less than or equal to 8% w/w and partially demineralized bone has a calcium content of greater than 8% w/w but less than the original calcium content of the bone.

The term “surface demineralized bone” as used herein refers to bones, bone segments, bone fragments, bone fibers or bone particulates which have been treated to reduce the calcium content of the surface of the bone, without significantly reducing the calcium content within the body of the segment, fragment, fiber or particulate.

The term “chaotropic agent” will be understood to mean a compound capable of disrupting hydrogen bonding between water molecules in a water solution to affect the stability of other molecules, and particularly macromolecules such as proteins, in the solution. Examples of chaotropic agents include urea, thiourea, guanidine thiocyanate, guanidine HCl and mixtures thereof.

The term “salt” as used herein will be understood to mean a chemical compound consisting of an ionic assembly of positively charged cations and negatively charged anions, which results in a compound with no net electric charge. Examples of suitable salts include calcium chloride, sodium chloride, or potassium chloride.

The term “bone growth factor” will be understood to include any growth factor occurring in bone, including, for example, bone morphogenic protein-2 (BMP-2), bone morphogenic protein-7 (BMP-7), transforming growth factor beta 1 (TGF-p1), platelet derived growth factor-BB (PDGF-BB), vascular endothelial growth factor (VEGF), and insulin like growth factor-2 (IGF-2).

The singular forms “a”, “an”, and “the” include plural forms unless the context clearly dictates otherwise. Thus, for example, reference to “a bone growth factor” includes reference to one or more of such elements.

It has been found that demineralized bone can be incubated with a solution comprising a chaotropic agent at a temperature in the range of about 2° C. to about 30° C. to release a bone growth factor into the solution. The chaotropic agent may then be selectively removed and the released growth factor is retained with the demineralized bone. It has further been found that this process can be conducted in the absence of added protease inhibitors.

The demineralized bone may include human bone or animal bone, such as bovine or porcine. The bone may include one or more types of bone, such as, cortical bone or cancellous bone. It may be in the form of whole bone, bone segments, bone fragments or it may be further processed by cutting and/or grinding into segments, blocks, wedges, chips, particles, fibers, cancellous strips/sponges or the like.

Demineralization can be carried out by any known method including for example by treatment with a strong acid such as HCl, a weak acid such as acetic acid or a chelating agent such as EDTA to remove calcium. In a particular embodiment, the demineralized bone is human bone that has been ground into particles and treated with HCl to remove calcium. In a further example the demineralized bone is fully demineralized bone matrix. In another embodiment, the demineralized bone is human bone that has been milled into fibers and treated with HCl to remove calcium.

In examples, the demineralized bone is a particulate having a particle size of about 0.025 mm to about 5 mm. In a further example the demineralized bone has a particle size of about 0.05 mm to about 4 mm, from about 0.1 mm to about 3 mm. In a further aspect the particle size is about 0.025 mm to 0.125 mm, in yet a further aspect the particle size is about 33 0.425 mm to about 1 mm, in yet a further aspect the particle size is about 2 mm to about 3 mm. Demineralized bone may be prepared or may be obtained from commercial sources.

The demineralized bone (DB) is treated by incubating the DB with a solution comprising a chaotropic agent. In one embodiment the solution is an aqueous solution. The solution of chaotropic agent may be in a concentration in the range of about 2M to about 8M. In a further aspect the concentration of the chaotropic agent in the solution is about 4M, about 6M or about 8M. The solution may include one or more chaotropic agents. The one or more chaotropic agents may be urea, thiourea, or guanidine HCl. In one embodiment the solution of a chaotropic agent is a solution of 6M urea.

The incubation is carried out a temperature between about 2° C. to about 30° C. or from about 12° C. to about 30° C., or from about 15° C. to about 25° C. In a particular embodiment, the incubation is carried out at ambient temperature in the range of about 18° C. to about 22° C. In a further aspect the incubation is carried out without added heating or cooling.

The incubation is carried out for a period of time sufficient to allow release of a growth factor from the demineralized bone. In an embodiment the incubation period is from about 2 hours to about 36 hours. In a further embodiment the incubation period is from about 4 hours to about 30 hours. In a further embodiment the incubation period is at least about 4, about 14, about 18, about 22 or about 30 hours. In a further embodiment the incubation period is about 4, about 14, about 18, about 22 or about 30 hours.

In one embodiment the incubation is carried out at a pH in the range of 5 to 8. In a further embodiment the pH range is 6 to 7.8. In still a further aspect the pH range is 6.5 to 7.5. In still a further embodiment the pH range is 6 to 7.

Following the incubation with the solution of chaotropic agent, the chaotropic agent is selectively removed to leave the released bone growth factor and demineralized bone.

In one embodiment selective removal of the chaotropic agent is carried out by filtration through a semi-permeable membrane. In one embodiment the semi-permeable membrane has a molecular weight cut off (MWCO) in the range of about 1 KDa to about 25 KDa or about 3.5 KDa to about 14 KDa. In a particular embodiment the MWCO is about 10 KDa. In another embodiment the MWCO is in the range of about 6 KDa to about 8 KDa.

In one example, the filtration through a permeable membrane is carried out by dialysis through a dialysis membrane. In another example the filtration is carried out by tangential flow filtration.

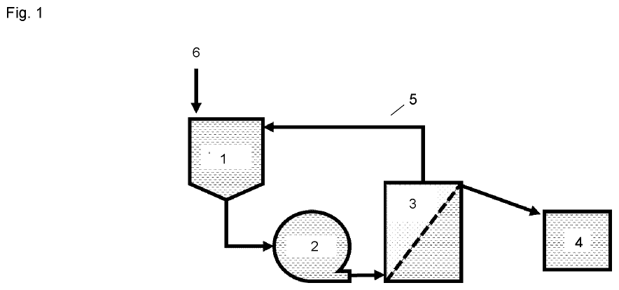

FIG. 1 depicts an example of a tangential flow filtration system. The figure shows a tank (1) which holds the bone matrix and the chaotrope solution which will contain solubilized proteins including bone growth factors. The figure also shows a pump (2) for pumping the chaotrope solution containing the solubilized proteins across a filter (3). The filter is selected to have a MWCO as described above. The system allows for selective removal of the permeate (4), which includes the chaotropic agent and salt, while retaining the retentate, which includes the released bone growth factors, which may optionally be returned back to the tank as indicated by (5). The permeate fluid volume can be replaced by the addition of water (6) to the tank

After removal of the chaotropic agent, the released bone growth factor and demineralized bone are retained. The solution containing the released bone growth factor and demineralized bone may be reassociated by removing water. In a particular example, the concentrate containing bone growth factor and demineralized bone may be lyophilized to remove remaining water.

In another embodiment the growth factors may be selectively separated from the chaotropic agent by precipitating the growth factors by adding ethanol or trichloroacetic acid (TCA) and then filtering or centrifuging followed by removing the supernatant from the DB and growth factors. Residual water and ethanol or TCA can then be removed from the DB and growth factors for example by lyophilization.

0053 In a further aspect it has been found that a salt may be added to the solution comprising the chaotropic agent to release a bone growth factor into the solution. Examples of suitable salts include inorganic salts. In a particular embodiment, the salt may include one or more of calcium chloride, sodium chloride, or potassium chloride. In a further embodiment the salt is calcium chloride and/or sodium chloride. In one embodiment the salt may be in a concentration in the range of about 0.1M to about 1M. In a further embodiment the salt may be in a concentration in the range of 0.25M to 0.75M. In a further embodiment the salt may be in a concentration of about 0.25M, 0.5M or 0.75M. In a particular embodiment the salt is in a concentration of about 0.5M.

In an embodiment the solution for incubating with the demineralized bone comprises, 6M urea and 0.5M CaCl₂.

In another aspect, it has been found that various growth factors are released from demineralized bone when treated by the processes described herein. For example, the bone growth factors released may include bone morphogenic protein (BMP), including for example, bone morphogenic protein-2 (BMP-2) and bone morphogenic protein-7 (BMP-7), transforming growth factor beta (TGF-ß1), platelet derived growth factor-BB (PDGF-BB), vascular endothelial growth factor (VEGF), insulin-like growth factor 2 (IGF-2) or others.

While prior methods for releasing bone growth factor have taught the need to include a protease inhibitor to prevent enzymatic degradation of released bone growth factors the inventors have found that release of bone growth factors can be carried out as described herein without the need to add protease inhibitors. The absence of protease inhibitors in this process provides the dual advantages of improving efficiency and cost effectiveness of the process by reducing the number of reagents and simplifying the process.

Further, while prior art processes for treating demineralized bone have required that the steps of incubation and removing of the chaotropic reagents occur at low temperatures, in the order of 2° C. to 4° C., the inventors have found that these steps can be conducted at ambient temperature in the temperature ranges described above. The feature of conducting the process at ambient temperature also provides significant advantages in terms of efficiency and cost effectiveness, as the process does not need to be carried out under refrigeration making the process both simpler and less expensive. Additionally, it has been found that the process carried out at ambient temperature results in a greater amount of BMP-7 and TGF-ß1 being released than when the process is conducted at low temperature in a fridge.

The processes described herein have been developed to enhance the bioavailability of BMPs of demineralized bone as a means of improving osteoinductive activity of the demineralized bone. The processes described herein can also enhances the bioavailability of other growth factors in demineralized bone, including, for example, TGF-ß1, VEGF, IGF-2 and PDGF-BB which may further improve the potential for bone repair by demineralized bone, by stimulating angiogenesis and osteogenesis.

The product of this process can be combined with mineralized cortical or cancellous particulate to produce a product that can be visible radiologically, which provides the clinician the ability to identify the product and its position within the body using X-rays, CT scans or other similar imaging modalities after placement.

The product of this process can be combined with a gel including such as those produced using poloxamer P407, or chitosan or hyaluronic acid or carboxymethyl cellulose to produce a flowable product. The flowable product can be delivered using a canula or straw or other suitable device.

The product of this process can also be combined with a cement including calcium phosphate cements (CPC) or calcium sulphate cements which can then be used where increased biomechanical strength is required.

The product of this process can be used to promote bone formation and/or bone fusion. The product can be used to treat boney voids, bone defects, non-union of bones, for example, fracture non-unions, or to promote fusion of 2 bones, such as vertebra or ankle bones. The product can be applied or implanted at the site where bone formation and/or bone fusion is desired to occur.

In another aspect, there is provided a product prepared by the process described herein. The product of the present process can be used to promote bone formation and/or to promote bone fusion. In particular, the product of the present processes can be used in the treatment of bone voids, bone defects, non-unions and promoting fusion of bones.

### EXAMPLES

### Example 1: Preparation of Bone

Bovine Bone: Femurs from calves were obtained from a slaughter house, cleaned of soft tissue and marrow, cut into blocks, soaked in 70% isopropyl alcohol and frozen. Bone particulate was prepared by grinding the blocks into various sizes using a hammer mill with various screens ranging from 3 mm to 0.3 mm. After grinding the bone was sieved to the desired particle size. Bone fibres were prepared from the frozen blocks by milling the bone using an end mill which was passed repeatedly across the block to produce mineralized fibres of the desired length and width. After grinding or milling the bone was stored at −30° C. until it was demineralized.

Human Bone: Mineralized and demineralized bone was obtained from AATB accredited tissue banks as cortical bone particulate, cancellous bone particulate, cancellous strips or cortical fibres.

Demineralization: Mineralized bone was demineralized by incubation with repeated changes of HCl solutions at various concentrations (0.5 to 1.2M) at room temperature with constant stirring over several minutes to days.

The bone was then rinsed with water and/or phosphate buffered saline (PBS) or 20 to 200 mM phosphate buffer (pH 7.2±0.2) to neutralize and remove residual acid.

The demineralized bone matrix slurry produced was either used immediately, held at 2° C. to 8° C. or frozen at −30° C. until ready for use.

When previously lyophilized demineralized bone obtained from tissue banks was used it was rehydrated with water prior to further processing.

### Example 2: Treatment of DBM with Solutions Containing 6 or 4M Urea with and without Calcium Chloride

2 batches of 100 g of bovine bone particles (420 to 1000 μm) from the same pool of ground bovine bone, prepared as described in Example 1 were processed as follows. For Batch 1 the bone was demineralized by 3×1 hour incubations with 0.5M HCl (1:10 w/vol). The bone matrix was then rinsed with water (1:10 g/mL) 5×20 minutes to produce demineralized bone matrix slurry.

For Batch 2 the bone was demineralized as for batch one, but after being rinsed with water 4 times, the matrix was then held overnight (approximately 18 hours) in a fifth water rinse at 2 to 8° C. without stirring.

The demineralized bone matrix slurry of Batch 1 was split into 4 aliquots of approximately 25 mLs and the demineralized bone matrix slurry of Batch 2 was split into 5 aliqouts of approximately 20 mLs. For each batch, 4 aliquots were transferred to previously hydrated dialysis membrane bags (MWCO 10KDa). These bags containing the matrix slurry were then incubated with stirring with various solutions (1:10 v/v) containing 4 or 6M urea with either no or 0.5M CaCl₂ (see Table 2.1). No enzyme inhibitors were added to these solutions and the incubation was performed between 18 and 25° C. overnight.

For Batch 2 the 5^{th} aliquot of the demineralized bone matrix (DBM) slurry was washed with water at 1:10 (v/v) dilution a further three times frozen and lyophilized.

The different treatments are summarized in Table 2.1

TABLE 2.1
Different treatments used in Example 2

| | | | Urea solution | |
|---|---|---|---|---|
| Batch | Group | | pH at time | Duration |
| # | # | Solution | of use | (hr) |
| Batch 1 | 1 | 6M Urea, 0.5M CaCl₂ | 6.97 | 18 |
| Batch 1 | 2 | 6M Urea | 7.52 | 18 |
| Batch 1 | 3 | 4M Urea, 0.5M CaCl₂ | 6.82 | 18 |
| Batch 1 | 4 | 4M Urea | 7.30 | 18 |
| Batch 2 | 5 | 6M Urea, 0.5M CaCl₂ | 6.91 | 18 |
| Batch 2 | 6 | 6M Urea | 7.81 | 18 |
| Batch 2 | 7 | 4M Urea, 0.5M CaCl₂ | 7.15 | 18 |
| Batch 2 | 8 | 4M Urea | 7.76 | 18 |
| Batch 2 | DBM | Additional water rinse | n/a | n/a |

After being incubated with the different solutions overnight the treatment solution was removed and the dialysis tube with each test article was dialyzed against 4 changes of water (1:20 v/v) each for 1 hour.

The slurries were recovered from the dialysis tubes, frozen and lyophilized.

To assess protein release and BMP bioavailability duplicate 500 mg samples from each group was placed in sterile containers to which 20 mL of 50 mM acetic acid (AcOH; pH 3.0 to 3.5) or Tris buffered saline (TBS; pH 7.0 to 7.5) was added. The bottles were agitated on an orbital shaker at room temperature for 27 hrs. The supernatant was collected, centrifuged at 2,700 rcf and duplicate aliquots were collected and frozen.

BMP-2 and BMP-7 content of the AcOH and TBS extracts was assessed using the hBMP-2 and hBMP-7 Quanitikine ELISA kits (R&D Systems, Minneapolis, MN) following the manufacturer's instructions.

Total protein was measured using a Coomassie Plus kit (Thermofisher Scientific, Toronto, Canada) with BSA as the standard.

Results were normalized to the amount of matrix extracted and the mean value for each group was determined as shown in Table 2.2.

### Results

The TBS extracts had significantly higher total protein and BMP-2 content than the acetic acid extracts of the same matrix.

When compared to DBM, the 6 and 4M urea-only treated matrices had lower BMP-2 levels in the TBS extracts and lower or similar BMP-2 levels in the AcOH extracts, while the BMP-7 levels were higher in the AcOH extracts.

The addition of 0.5M calcium chloride increased the amount of total protein and BMP-2 concentrations in the AcOH and TBS extracts. The BMP-7 levels in the AcOH extracts were also increased.

Compared to DBM both batches of 6M urea, 0.5M CaCl₂ treated matrix had higher BMP-2 concentrations in the TBS (1245 and 794 vs 543 ng/g) and the AcOH extract (3.86 and 3.20 vs 0.22 ng/g).

TABLE 2.2
Protein and BMP concentration in extracts

| | | | AcOH extract | | | TBS extract | |
|---|---|---|---|---|---|---|---|
| | | | Total | | | Total | |
| Batch | Group | Incubation | protein | BMP-2 | BMP-7 | protein | BMP-2 |
| # | # | Solution | (mg/g) | (ng/g) | (ng/g) | (mg/g) | (ng/g) |
| 1 | 1 | 6M Urea, 0.5M CaCl₂ | 3.65 | 3.86 | 25.89 | 11.58 | 1.25 |
| 1 | 2* | 6M Urea | 1.47 | 0.04 | 1.57 | 6.94 | 0.22 |
| 1 | 3 | 4M Urea, 0.5M CaCl₂ | 1.98 | 0.96 | 10.27 | 10.10 | 0.77 |
| 1 | 4 | 4M Urea | 1.15 | 0.26 | 0.84 | 5.77 | 0.33 |
| 2 | 5 | 6M Urea, 0.5M CaCl₂ | 3.67 | 3.20 | 22.37 | 11.39 | 0.79 |
| 2 | 6 | 6M Urea | 2.24 | 0.19 | 4.65 | 6.86 | 0.19 |
| 2 | 7 | 4M Urea, 0.5M CaCl₂ | 2.74 | 0.80 | 15.35 | 9.15 | 0.26 |
| 2 | 8 | 4M Urea | 1.38 | 0.06 | 1.23 | 7.27 | 0.06 |
| 2 | DBM-1 | additional water rinse | 0.30 | 0.22 | <LLD | 11.11 | 0.54 |

*the dialysis bag for group 2 appeared to have leaked as particles of matrix was observed in the incubation solution and the amount of slurry collected (18mL) was reduced compared to the amounts from the other bags in Batch 1 (~25 mL).
“<LLD” = below limit of detection

### Discussion

Treatment with 4 or 6M urea alone reduces the amount of BMP-2 in TBS extracts compared to extracts of DBM. The addition of 0.5M CaCl₂ increased the level of BMP-2 in the TBS and AcOH extracts. Only treatment with 6M urea, 0.5M CaCl₂ consistently increased BMP-2 levels compared to extracts of DBM.

A similar pattern was observed in acetic acid extracts, for both BMP-7 and BMP-2.

These results demonstrated that even without the use of protease inhibitors or extraction of BMPases when samples were treated and dialyzed at ambient temperature rather than in the cold, treatment of DBM with 6M urea solutions containing 0.5M CaCl₂ reproducibly increased the amount of BMP released into solutions at various pHs.

### Example 3: Varying Urea and Calcium Chloride Concentrations

2 batches of 50 g of bovine bone (particle size 420 to 1000 μm) from the same pool of ground bovine bone, prepared as described in Example 1, were demineralized with 3 incubations with 0.5M HCl for 60 to 90 minutes each, with agitation at room temperature. The acid was then removed and the DBM slurry rinsed with 8-9 rinses of water (1:10 v/v) at room temperature with agitation until the pH of the water after 10 minutes was >6.5 pH

The demineralized bone matrix slurry from each batch was split into 4 fractions, approximately 10 to 12.5mLs each and transferred to previously hydrated dialysis membrane bags (MWCO 10KDa). These bags containing the matrix slurry were then incubated with stirring with various solutions (1:10 v/v) containing 4, 6 or 8M urea with either 0.25, 0.5 or 0.75M CaCl₂. No enzyme inhibitors were added to these solutions and the incubation was performed at room temperature overnight.

TABLE 3
Different treatments used in Example 3

| Batch | Group | Urea-calcium chloride |
|---|---|---|
| # | # | Concentrations (M) |
| 2 | 1 | 8M urea, 0.75M CaCl₂ |
| 1 | 2 | 8M urea, 0.25M CaCl₂ |
| 2 | 3 | 6M urea, 0.75M CaCl₂ |
| 2 | 4 | 6M urea, 0.5M CaCl₂ |
| 1 | 5 | 6M urea, 0.25M CaCl₂ |
| 1 | 6 | 4M urea, 0.75M CaCl₂ |
| 2 | 7 | 4M urea, 0.25M CaCl₂ |
| 1 | 8-DBM | additional water rinse |

The dialysis bags were immersed in the various urea-calcium chloride solutions, with agitation at room temperature for 19 hours 10 minutes (Batch 1) or 18 hours 25 minutes (Batch 2).

The urea-calcium chloride solution was then removed, and the bags rinsed with water (1:10 v/v) with agitation at room temperatures for 60±15 minutes. The water was then removed and 3 more 60±15 minute water rinse steps done (total of 4 rinses). The matrix slurry in the dialysis bags was recovered, frozen and lyophilized.

Following lyophilization 250 mg of the various matrices were extracted with 20 mL of 50 mM acetic acid or tris buffered saline for 7 days with agitation at room temperature. At 1, 4 and 7 days the samples were centrifuged at 2,700 rcf and 1 mL was removed. The samples were returned to the shaker and the 1 mL aliquots were frozen for later analysis.

The BMP-7 concentration in the extract samples was analyzed using a Quanitikine ELISA as described in example 2. The results were normalized to the starting weight of the matrix. Results are shown in Table 3.2.

### Results

The BMP-7 concentration in the AcOH samples were lower than the concentrations in the corresponding TBS extracts. The AcOH results showed, with the exception of the DBM (59% increase) and 4M urea, 0.25M CaCl₂ (31%) matrices, only a small increase (<10%) or a decrease (−19%) in BMP-7 levels between Day 1 and Day 7.

The TBS extracts showed an increase in BMP-7 concentration between Day 1 and for all groups (20 to 89%).

All the matrices, with the exception of Group 7 (4M urea, 0.25M CaCl₂), had higher levels of BMP-7 in the AcOH extracts compared to DBM (ranging from 270 to 450% higher)

On day 1 all the TBS extracts of matrices treated with solutions containing 6 or 8M urea had higher concentrations of BMP-7 (on average being 33% higher), while those containing 4M urea were 36% lower. For the day 7 the average difference for the TBS extracts of the 6 and 8M matrices compared to DBM was 5% higher while the 4M urea treated matrices were 25% lower than for the TBS extracts of DBM.

TABLE 3.2
BMP-7 (ng/g) in extracts of matrices prepared in Example 3

| Batch | Group | Urea-calcium chloride | AcOH | | | TBS | | |
|---|---|---|---|---|---|---|---|---|
| # | # | Concentration | 1 DY | 4 DY | 7 DY | 1 DY | 4 DY | 7 DY |
| 2 | 1 | 8M urea, 0.75M CaCl₂ | 1.84 | 1.79 | 1.90 | 14.81 | 17.79 | 18.26 |
| 1 | 2 | 8M urea, 0.25M CaCl₂ | 1.73 | 2.12 | 1.93 | 15.39 | 17.71 | 19.87 |
| 2 | 3 | 6M urea, 0.75M CaCl₂ | 1.94 | 2.04 | 1.96 | 16.61 | 20.27 | 23.07 |
| 2 | 4 | 6M urea, 0.5M CaCl₂ | 2.42 | 2.38 | 2.05 | 13.64 | 15.24 | 18.59 |
| 1 | 5 | 6M urea, 0.25M CaCl₂ | 2.44 | 2.36 | 2.25 | 15.45 | 17.71 | 20.55 |
| 1 | 6 | 4M urea, 0.75M CaCl₂ | 1.55 | 1.39 | 1.28 | 10.11 | 16.95 | 16.40 |
| 2 | 7 | 4M urea, 0.25M CaCl₂ | 0.20 | 0.24 | 0.28 | 4.65 | 8.78 | 12.22 |
| 1 | 8-DBM | Additional water rinse | 0.38 | 0.49 | 0.69 | 11.45 | 16.84 | 19.14 |

### Discussion

These results demonstrate that demineralized bone matrix treated with 6M or 8M urea containing 0.25 to 0.75M CaCl₂ increases the release (bioavailability) of BMPs from the matrix into 50 mM acetic acid and into TBS.

### Example 4: Effect of Using Other Chaotropes and Other Salts on Bioavailability of BMPs

Two batches of 60 g of bovine bone (particle size 74 to 420 μm) from the same pool of bone, prepared as described in Example 1 were demineralized by 3×1-hour incubations with 0.5M HCl (1:10 w/vol). The bone matrix was then rinsed with water (1:10 g/vol) 8× for 10 minutes, until the pH of the water is greater than pH 6.0.

Each batch was split into 5 portions and was transferred to previously hydrated dialysis membrane (regenerated cellulose, MWCO 10 kDa) and was then incubated with various chaotrope and salt solutions (C/S solutions) containing a chaotrope (either 6M urea or 4M Guanidine HCl (Gdn·HCl)) and an inorganic salt (0.5M CaCl₂, MgCl₂, KCl, NaCl) overnight at room temperature with stirring. The Guanidine HCl containing solutions were prepared in 20 mM Tris (pH 7.2). No enzyme inhibitors were added to these solutions and the pH was not adjusted after addition of the chaotropes or salts. The different treatments are summarized in Table 4.1.

TABLE 4.1
Different treatments used in Example 4

| | | | C/S solution | |
|---|---|---|---|---|
| Batch | Group | | pH at time | Duration |
| # | # | C/S Solution | of use | (hr) |
| Batch 1 | 1 | 6M Urea, 0.5M CaCl₂ | 6.84 | 18.5 |
| Batch 1 | 2 | 6M Urea, 0.5M MgCl₂ | 7.20 | 18.5 |
| Batch 2 | 3 | 6M Urea, 0.5M NaCl | 7.81 | 19.0 |
| Batch 2 | 4 | 6M Urea, 0.5M KCl | 7.83 | 19.0 |
| Batch 2 | 5 | 4M Gdn•HCl/20 mM Tris, 0.5M CaCl₂ | 6.82 | 19.0 |
| Batch 1 | 6 | 4M Gdn•HCl/20 mM Tris | 6.54 | 18.5 |
| Batch 1 | 7 | 4M Gdn•HCl/20 mM Tris, 0.5M NaCl | 6.78 | 18.5 |
| Batch 2 | 8 | 4M GdnHCl/20 mM Tris, 0.5M KCl | 6.79 | 19.0 |
| Batch 1 | DBM-1 | Water | n/a | 18.5 |
| Batch 2 | DBM-2 | Water | n/a | 19.0 |

After being incubated with the different solutions overnight the treatment solution was removed and the dialysis tube with each test article was rinsed with 4 changes of water (1:20 v/v) each for 1 hour. The slurries were recovered from the dialysis tubes, frozen at −30° C. and lyophilized.

To assess protein release and BMP bioavailability 500±10 mg of matrix from each group was placed in PETG Media Bottles to which 20 mL of 50 mM acetic acid was added. The bottles were agitated on an orbital shaker at room temperature. At 2 days the supernatant was collected, centrifuged at 2,700 rcf and duplicate 500 μL aliquots were collected and frozen.

Total protein in the AcOH extract was assessed using the Pierce Coomassie Plus Kit (Thermofisher) following the manufacturer's instructions. The total protein was determined by comparing to a standard curve produced using BSA.

BMP-2 content of the AcOH extract at 2 days was assessed using the hBMP-2 quanitikine ELISA (R&D Systems, Minneapolis, MN) following the manufacturer's instructions. Results were normalized to the amount of matrix extracted.

To assess the Matrix pH 60±5 mg of matrix from each group was mixed with 4 mL of 0.9% saline solution, vortexed briefly and allowed to incubate for 10-20 minutes. The pH of the supernatant was then measured.

### Results

The DBM samples had undetectable levels of BMP-2 in the AcOH extracts.

Extracts of matrices treated with 6M urea containing CaCl₂, NaCl or KCl had BMP-2 levels between 2.3 and 5.7ng/g.

Extracts of matrices treated with 4M Gdn·HCl/20 mM Tris alone, or with the addition of 0.5M NaCl had BMP levels between 4.9 and 5.5ng/g.

Extracts of matrices treated with 6M Urea, 0.5M MgCl₂ or with 4M Gdn·HCl/20 mM Tris when either CaCl₂ or KCl was added had undetectable levels of BMP-2.

TABLE 4.2
Results from Example 4

| | | | Ptn | BMP-2 |
|---|---|---|---|---|
| Group | | Matrix | 2 dy | 2 dy |
| # | Treatment | pH | (μg/g) | (ng/g) |
| 1 | 6M Urea, 0.5M CaCl₂ | 7.12 | 3,766 | 2.29 |
| 2 | 6M Urea, 0.5M MgCl₂ | 6.97 | 3,781 | <LLD |
| 3 | 6M Urea, 0.5M NaCl | 7.23 | 3,363 | 3.50 |
| 4 | 6M Urea, 0.5M KCl | 7.21 | 4,365 | 5.71 |
| 5 | 4M Gdn•HCl/20 mM Tris, | 7.01 | 2,080 | <LLD |
| | 0.5M CaCl₂ | | | |
| 6 | 4M Gdn•HCl/20 mM Tris | 7.00 | 5,044 | 4.91 |
| 7 | 4M Gdn•HCl/20 mM Tris, | 7.30 | 4,012 | 5.49 |
| | 0.5M NaCl | | | |
| 8 | 4M GdnHCl/20 mM Tris, | 7.18 | 3,004 | <LLD |
| | 0.5M KCl | | | |
| DBM-1 | Water | 6.89 | 685 | <LLD |
| DBM-2 | Water | 6.96 | 757 | ND |

“<LLD” = below limit of detection
“ND” = not done

### Discussion

4M Gdn·HCl/20 mM Tris with or without 0.5M NaCl increased the amount of BMP-2 in the AcOH extracts compared to DBM; however the addition of CaCl₂ or KCl to the 4M Gdn·HCl/20 mM Tris resulted in undetectable levels, equivalent to the DBM control.

As previously described treatment of DBM slurry with 6M urea, 0.5M CaCl₂ results in an increase in BMP-2 levels in acetic acid extracts compared to DBM. While the replacement of CaCl₂ with MgCl₂ did not result in a similar increase compared to DBM the use of 0.5M NaCl or KCl as the salt resulted in even higher BMP-2 levels.

These results demonstrate that demineralized bone matrix treated with 6M urea containing 0.5M CaCl₂, NaCl or KCl increases the release of BMPs from the matrix

These results demonstrate that demineralized bone matrix treated with 4M Guanidine HCl with or without the addition of 0.5M NaCl increases the release of BMPs from the matrix.

### Example 5: Effect of Duration of Treatment and Particle Size on Bioavailability of BMPs

Bovine bone particulates were prepared from a single pool of calf femurs that was cleaned, cut into segments, ground and passed through 3 mm screen.

Particulates sized 2-3 mm were prepared by sieving the ground bone and retaining the particulates captured using a 2 mm sieve.

Particulates 0.42 to 1 mm were prepared by sieving the bone and retaining the particulates that passed through a 1 mm sieve and that was retained in the 0.42 mm sieve. The particulates sized 0.42 to 1 mm were demineralized as described in Example 2. The particulates sized 2-3 mm were demineralized with 6 changes of acid over 46 hours. Both groups of particles were then rinsed with water until the pH of the rinse was increased to >6.5 and the excess water removed.

The 2 matrix slurries produced (approximately 1 mL per g/mineralized particulate) were then each split into 5 aliquots, 4 of which transferred to hydrated dialysis tubing (MWCO 10 KDa) and incubated with 6M urea, 0.5M CaCl₂ at room temperature with agitation for between 4 and 30 hours as summarized in table 5.1 with the 5th aliquot being immediately transferred to a 50 mL centrifuge tube and frozen at −30° C. as a DBM control.

After incubation with the C/S solution the matrices were dialyzed against 4 changes of water (1:20 v:v) 1 hour per change and the conductivity of the water monitored. The slurries were then transferred to a centrifuge tube and frozen at −30° C.

All frozen matrices were then lyophilized.

Samples from each group were evaluated for release of BMP-7 into 50 mM acetic acid (1 day) or TBS (up to 14 days) as described in Example 3.

Samples from each group were assessed for matrix pH as described in Example 4

Samples from each group were assessed for matrix conductivity by extracting duplicate 300 mg samples of each matrix with laboratory grade water for 18 hours in a 15 mL centrifuge tubes at room temperature on a rocker. The centrifuge tubes were then centrifuged at 2,700 rcf for 10 minutes to pellet the matrix and the supernatant was collected and its conductivity measured.

TABLE 5.1
Different treatments used in Example 5

| | | | | pH of C/S | | |
|---|---|---|---|---|---|---|
| Group | | | | solution | Duration | |
| # | Particle size | | C/S Solution | when used | (hr) | |
| 1 | 0.425-1 | mm | 6M urea, 0.5M CaCl₂ | 7.14 | 4 | hr |
| 2 | 0.425-1 | mm | 6M urea, 0.5M CaCl₂ | 7.12 | 14 | hr |
| 3 | 0.425-1 | mm | 6M urea, 0.5M CaCl₂ | 7.12 | 18 | hr |
| 4 | 0.425-1 | mm | 6M urea, 0.5M CaCl₂ | 7.12 | 22 | hr |
| 5 | 0.425-1 | mm | n/a | n/a | 0 | hr |
| 6 | 2-3 | mm | 6M urea, 0.5M CaCl₂ | 7.17 | 14 | hr |
| 7 | 2-3 | mm | 6M urea, 0.5M CaCl₂ | 7.17 | 18 | hr |
| 8 | 2-3 | mm | 6M urea, 0.5M CaCl₂ | 7.17 | 22 | hr |
| 9 | 2-3 | mm | 6M urea, 0.5M CaCl₂ | 7.17 | 30 | hr |
| 10 | 2-3 | mm | n/a | n/a | 0 | hr |

“n/a” = not applicable

### Results

Acetic acid extracts of DBM particulate (0.42 to 1 mm) had undetectable levels of BMP-7, while the 2-3 mm DBM particulates had low but measurable amounts of BMP-7 (3 ng/g).

Particulates treated with C/S solution had BMP-7 levels of between 13 and 50 ng/g, with the amount increasing with increasing duration of treatment up to 22 hours.

The matrix pH of the C/S treated samples was between 0.5 to 1.3 pH units higher than the value for the matching DBM. The matrix pH of the larger particulates, which had been demineralized for 46 hours was on average 2.0 pH units lower than the corresponding smaller particulates which were only demineralized for 3 hours.

TABLE 5.2
Summary of Results for Example 5

| | Particle | | | Matrix | AcOH | | | | | |
|---|---|---|---|---|---|---|---|---|---|---|
| | size | Duration | Matrix | conductivity | BMP-7 | TBS BMP-7 (ng/g) | | | | |
| Gp# | (mm) | (hr) | pH | (μS/cm) | (ng/g) | 1 | 4 | 7 | 11 | 14 |
| 1 | 0.42-1.0 | 4 | 7.03 | 79 | 34 | 51.9 | 99.2 | 165.7 | 179.5 | 213.5 |
| 2 | 0.42-1.0 | 14 | 6.70 | 118 | 33 | 43.4 | 80.3 | 125.5 | 153.8 | 153.4 |
| 3 | 0.42-1.0 | 18 | 6.53 | 78 | 43 | 94.5 | 141.5 | 169.4 | 215.3 | 197.5 |
| 4 | 0.42-1.0 | 22 | 6.48 | 126 | 46 | 45.0 | 76.8 | 114.8 | 143.8 | 138.6 |
| 5 | 0.42-1.0 | 0 | 5.98 | 123 | <LLD | 19.2 | 46.2 | 88.5 | 123.1 | 129.7 |
| 6 | 2-3 | 14 | 5.03 | 368 | 13 | 6.3 | 9.0 | 8.6 | 11.1 | 13.6 |
| 7 | 2-3 | 18 | 4.53 | 509 | 39 | 14.8 | 11.3 | 9.6 | 13.1 | 11.7 |
| 8 | 2-3 | 22 | 4.41 | 527 | 50 | 26.2 | 18.1 | 19.5 | 17.3 | 18.9 |
| 9 | 2-3 | 30 | 4.92 | 196 | 35 | 16.9 | 22.3 | 25.8 | 27.2 | 36.7 |
| 10 | 2-3 | 0 | 3.78 | 1175 | 3 | 0.8 | <LLD | 1.9 | 1.5 | <LLD |

“<LLD” = below limit of detection
“ND” = not done

### Discussion

Treatment with the C/S solution increased the amount of BMP-7 released into the AcOH and TBS extracts of 0.42 to 1 mm and 2 to 3 mm particulates.

Even treatment times as short as 4 hours was sufficient to increase the amount of BMP-7 released into acetic acid or TBS.

From the results of examples 2 to 5 it is observed that the DBM has very low or undetectable levels of BMP-2 and BMP-7 in the AcOH extracts, while the NMP samples produced with 6M urea with a suitable salt or guanidine HCl with a suitable salt have measurable levels at least 4 and usually 10+ fold higher than DBM produced from the same bone. Further the amount of BMP-2 and 7 released from NMP after a 24 AcOH does not significantly increase with extended incubation in AcOH. In contrast, the DBM does have measurable levels of BMP-2 and BMP-7 in the TBS extracts although they are lower than in the NMP samples. Further the amount of BMP-7 released from the DBM and the NMP continues to increase with incubation with TBS for at least 14 days. TGF-ß1, VEGF and PDGF-BB are also seen to be elevated in AcOH extracts of NMP compared to DBM. These results suggest that the BMPs and possibly growth factors that are released by incubation with AcOH and with TBS come from 2 separate compartments in the matrix, a low pH extractable compartment and a neutral pH extractable compartment.

To further investigate the AcOH and TBS extractable pools, DBM and NMP produced from the DBM by incubation with 6M urea/0/5M CaCl₂ were either incubated in 50 mM acetic acid (250 mg in 20 mL) for 1 day at room temperature or were incubated with TBS (250 mg in 20 mL) at room temperature on an orbital shaker for 11 days. The samples extracted with TBS were then separated from the TBS by centrifugation and were extracted with AcOH for 1 day. The amount of BMP-7 in each extract was determined by ELISA. The 11 day TBS extract of NMP contained 173ng/g BMP-7 while the 11 day TBS extract of DBM contained 101ng/g BMP-7. The amount of BMP-7 in the pre-TBS AcOH extract was 7.3 and 1.2ng/g for NMP and DBM respectively while the amount of BMP-7 in the post-TBS AcOH extract was 10.7 and 2.1ng/g. These results indicate that the BMP-7 in the AcOH extractable BMP-7 is not depleted by incubation with TBS, but may actually increase.

It is generally accepted that incubation of demineralized bone with urea or guanidine HCl disrupts non-covalent bonds resulting in the release of non-collagenous proteins from the insoluble demineralized collagenous matrix. With the removal of the urea or guanidine these proteins once again interact with one another and with the collagenous matrix forming complexes. The inventors have previously reported that when bone is extracted with 6M urea 0.5M calcium chloride and the extract is separated from the matrix and dialysed with water a precipitate forms which can be collected by ultracentrifugation. This precipitate possesses BMP activity, which acts slowly on cells when added to their media in vitro. When that insoluble complex is incubated with 0.1% TFA the BMP activity is solubilized and when the TFA extract is added to culture media the response of the cells is more rapid and then declines when the media is removed and replaced with fresh media without the TFA extract, while the precipitate remains in the culture and the cellular response increases overtime (Peel et al., J. Craniofacial Surg (2003) 14:284-291), indicating that the BMPs present in the insoluble protein complex are rapidly released by exposure to low pH, while incubation of the precipitate with cell culture media results in a much slower release. Thus, it is likely that incubation of demineralized bone matrix with 6M urea, 0.5M calcium chloride followed by removal of the chaotrope and salts resulted in the formation of an insoluble protein complex incorporating some of the BMP (and possibly other growth factors) that was previously bound to the collagenous matrix and these complexed BMPs are released slowly when incubated in a neutral pH solution but rapidly when incubated with an acidic pH solution. Following implantation of the NMP as the matrix is resorbed the acidic environment would increase the rate of release of BMP into the local environment stimulating bone repair.

This acid extractable compartment may have existed in the mineralized bone matrix prior to demineralization; however, as the bone matrix was demineralized by incubation with acid the BMPs in the low pH extractable compartment would become soluble and diffuse into the acid, resulting in the low to undetectable levels of BMPs remaining in the low pH compartment.

To estimate the amount of BMP that is lost from the acidic compartment during demineralization the present inventors produced NMP wherein the bone matrix was demineralized in dialysis tubing and then the tubing was incubated with 6M urea 0.5M CaCl₂ and compared it to NMP produced at the same time from the same bone matrix, but the matrix was demineralized by stirring in HCl and then rinsed before transfer to the dialysis bag for treatment with 6M urea 0.5M CaCl₂. The 2 NMPs were then extracted with 50 mM AcOH for 1 day and the BMP-7 content measured by ELISA. The AcOH extracts of the NMP demineralized in the dialysis tubing contained 24ng/g BMP-7 and the extracts of the NMP produced from the bone demineralized as usual contained 4ng/g BMP-7.

While not wishing to be bound by theory, as the inventors have also observed increased release of BMPs into the TBS buffer, the treatment of the matrix with the chaotropic agent is believed to have resulted in a weakening in the binding of the BMPs to the insoluble collagenous matrix increasing their bioavailabilty when the matrix is exposed to neutral pH environment. Consequently, when the NMP matrix is initially implanted the BMPs in the neutral pH compartment would be released more rapidly than from the DBM matrix resulting in increased osteoinductive activity.

### Example 6: Effect of 6M Urea, 0.5M CaCl₂ Treatment on Human Cortical Particulate, Cortical Fibres, and Cancellous Strips

For the first study human mineralized cortical particulate (0.25-1 mm), mineralized cortical particulate (1-4 mm), mineralized cancellous particulate (1-4 mm) demineralized cancellous strips (˜16×20×48 mm) or demineralized bone fibres were obtained from various tissue banks. The mineralized cancellous and cortical particulate was demineralized as described in Example 2. The demineralized fibres and sponges were rehydrated with water.

The various test articles were each split into 2 aliquots. The first aliquot was transferred to a 50 mL centrifuge tube and frozen immediately and the second was placed in dialysis tubing and incubated with a chaotrope and salt solution (C/S solution) of 6M urea and 0.5M CaCl₂ at room temperature with agitation overnight. The C/S solution was removed and the matrix in dialysis tubing was rinsed with 4 changes of water over 4 hours. The slurry was removed from each dialysis tubing, transferred to a 50 mL centrifuge tube and frozen. The frozen aliquots were then lyophilized. The matrix produced following treatment with C/S solution is referred to as Natural Matrix Protein (NMP).

In a second study human mineralized cortical particulate (0.025 mm to 0.125 mm) was obtained from 3 separate donors. The particulates were demineralized as described in Example 2. The DBM slurry from each donor was split into 2 aliquots. The first aliquot was frozen and lyophilized. The second aliquot was treated with 6M urea and 0.5M CaCl₂ (C/S solution) as described in Example 2 to produce NMP slurry. The NMP slurry was then frozen and lyophilized.

Samples from each group were evaluated for release of BMP-7 into 50 mM acetic acid (1 day) or TBS (1 day) as described in Example 3. The acetic acid (AcOH) extracts were assayed for BMP-2, BMP-7, TGF-ß1, PDGF-BB and VEFG using Quantikine ELISA kits (R&D Systems).

### Results

The results are for the first study are summarized in Table 6.1 and the results for the second study are summarized in Table 6.2.

TABLE 6.1
Release of bone growth factors from matrices prepared in Example 6, Study 1.

| | | | | | TBS | AcOH | AcOH | AcOH | AcOH | AcOH |
|---|---|---|---|---|---|---|---|---|---|---|
| | form | C/S | C/S | Matrix | BMP-7 | BMP-7 | BMP-2 | TGF-β1 | PDGF | VEGF |
| ID# | (mm) | Rx | pH | type | (ng/g) | (ng/g) | (ng/g) | (ng/g) | (ng/g) | (ng/g) |
| S1 | Fiber | N | n/a | DBM | 7.70 | 0.26 | <LLQ | 0.39 | 0.49 | <LLQ |
| S2 | Fiber | Y | 7.44 | NMP | 29.49 | 10.50 | 9.77 | 3.24 | 2.00 | 0.35 |
| S3 | Cortical | N | n/a | DBM | ND | <LLD | <LLQ | 0.44 | 0.93 | 0.17 |
| | 1-4 mm | | | | | | | | | |
| S4 | Cortical | Y | 7.44 | NMP | ND | 1.18 | 2.45 | 3.70 | 2.33 | 0.91 |
| | 1-4 mm | | | | | | | | | |
| S5 | Cancellous | N | n/a | DBM | <LLD | <LLD | <LLD | ND | ND | ND |
| | 1-4 mm | | | | | | | | | |
| S6 | Cancellous | Y | 6.87 | NMP | 1.18 | 1.24 | <LLD | ND | ND | ND |
| | 1-4 mm | | | | | | | | | |
| S7 | Cortical | N | n/a | DBM | ND | <LLD | ND | ND | ND | ND |
| | 0.25-1 mm | | | | | | | | | |
| S8 | Cortical | Y | 7.15 | NMP | ND | 7.55 | ND | ND | ND | ND |
| | 0.25-1 mm | | | | | | | | | |
| S9 | Cancellous | N | n/a | DBM | ND | <LLD | ND | ND | ND | ND |
| | strip | | | | | | | | | |
| S10 | Cancellous | Y | 6.82 | NMP | ND | 1.13 | ND | ND | ND | ND |
| | strip | | | | | | | | | |

“ND” = not done
“n/a” = not applicable
“<LLD” = below lower limit of detection of the assay;
“<LLQ” = below the lower limit of quantitation of the assay

For samples from Study 2, the BMP-2 levels released from DBM into 50 mM acetic acid ranged from undetectable to 0.06ng/g, while the amount of BMP-2 released from NMP ranged from 0.31 to 6.68 ng/g. The BMP-7 levels released from DBM into 50 mM acetic acid ranged from 0.6 to 3.11 ng/g, while the amount of BMP-7 released from NMP ranged from 5.2 to 18.5 ng/g.

TABLE 6.2
Release of BMP-2 and BMP-7 from matrices
prepared as part of Example 6 Study 2.

| | Tissue | BMP-7 | BMP-2 |
|---|---|---|---|
| Donor | Type | ng/g | ng/g |
| D1 | DBM | 3.1 | <LLD |
| | NMP | 18.5 | 0.31 |
| D2 | DBM | 0.9 | <LLD |
| | NMP | 19.9 | 6.68 |
| D3 | DBM | 0.6 | 0.06 |
| | NMP | 5.2 | 4.53 |

“<LLD” below lower limit of detection

### Discussion

Treatment of human demineralized bone matrix (DBM) with C/S solution to produce NMP no matter the form of bone (cortical or cancellous particulate, cancellous strips or cortical fibres) increased the amount of BMP-2, BMP-7, TGF-ß1, PDGF-BB and VEGF released from the matrix.

Even after lyophilization treatment of DBM with C/S solution will result in increases in the amount of BMPs and other growth factors from the matrix.

### Example 7: In Vitro Osteoinductive Activity of NMP Compared to DBM

Lyophilized bovine bone particulate (0.425-1 mm) from the same pool of calf femurs was demineralized as described in Example 2 to produce DBM or was demineralized and then treated with 6M urea, 0.5M CaCl₂ C/S solution as described in Example 3 to produce NMP.

Inactive DBM was prepared by treating DBM slurry with three incubations of 4M GdnHCl 0.1M Tris pH 7.0 over 18 to 20 hours, followed by a water rinse, treatment with 26.5 mM acetic acid for 60-90 minutes followed by rinses with phosphate buffer and water after which the inactive DBM slurry was frozen and lyophilized.

The lyophilized particulates of DBM, NMP and inactive DBM (IDBM) were then weighed out and placed into #5 gelatin capsules (20 mg/capsule) and sterilized over chloroform vapors overnight.

The osteoinductive activity of the DBM, IDBM and NMP was determined using C2C12 cells as follows. The particulate in the gelatin capsules were aseptically mixed with DMEM+15 FBS % (20 mg/mL) and pipetted into wells of a 24 well tissue culture plate (250 μL/well). C2C12 cells were seeded into 24 well plates at 50,000 cells/well. Controls included cells only and cells incubated with 100ng/well rhBMP-2. An additional 200 μL/well of assay media was added every 2-3 days until day 9.

On day 9 the media was removed the cells were washed with cold TBS and then lysed by adding 0.5 mL CelLytic M to each well and frozen at −20° C.

The cell lysate was then assayed for alkaline phosphatase activity using paranitrophenol phosphate as described in Peel et al., J. Craniofacial. Surg. (2003) 14:284-291).

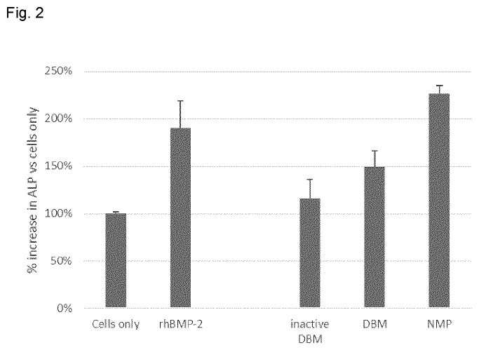

The results are summarized in FIG. 2 which is a bar graph showing the percent increase of alkaline phosphatase (ALP) for untreated C2C12 cells and C2C12 cells incubated with each of rhBMP-2, inactive DBM, DBM and NMP.

Cells exposed to NMP had significantly increased alkaline phosphatase activity compared to cells only, cells treated with inactive DBM or cells treated with active DBM (P<0.05, 1 Way ANOVA with holm sidak post hoc testing for all comparisons).

### Example 8: Effect of Temperature

Lyophilized bovine bone particulate (0.25-1 mm) from the same pool of calf femurs were demineralized as described in Example 1. Equal volumes of the demineralized matrix slurry was then loaded into 6 separate lengths of dialysis tubing. 3 dialysis tubes were incubated on a stirrer plate with 6M urea, 0.5M CaCl₂ (C/S solution) in a fridge (temperature range 2° C. to 8° C.) and the other 3 were stirred at ambient temperature (temperature range 20° C.-24° C.) overnight (19 hours) followed by repeated rinses with laboratory grade water. The temperature of the C/S solution was monitored using a digital temperature monitor.

The matrix recovered from the dialysis tubing (NMP) was then lyophilized. Following lyophilization duplicate samples of each test article was incubated with 50 mM acetic acid or tris buffer saline (TBS) for 24 hours after which the amount of BMP-7 in the extracts was measured by ELISA as described in Example 3.

A second study was performed where human bone was processed in a clean room operating at an ambient temperature 18±1° C. Demineralized microparticulate (0.25 to 1 mm) demineralized fibers from 4 donors were processed into NMP using 6M urea, 0.5M CaCl₂ (C/S solution) as described in Example 2. The NMP was then lyophilized and sterilized by gamma irradiation. Following irradiation samples of each test article were incubated with 50 mM acetic acid or tris buffer saline for 24 hours after which the amount of BMP-7, PDGF-BB, TGF-ß1 or IGF-2 in the extracts was measured by ELISA as described in Example 3.

### Results

The results of the first study are summarized in table 8.1 and of the second study are summarized in table 8.2

Table 8.1 BMP-7 and TGF-ß1 extracted from bovine NMP processed at different temperatures.

TABLE 8.1
BMP-7 and TGF-β1 extracted from bovine NMP processed at different temperatures.

| | C/S | | BMP-7 | | BMP-7 | | TGF-β1 | | TGF-β1 | |
|---|---|---|---|---|---|---|---|---|---|---|
| | temp | Test | AcOH extract | | TBS extract | | AcOH extract | | TBS extract | |
| | range | Article | (ng/g) | | (ng/g) | | (ng/g) | | (ng/g) | |
| GROUP | (° C.) | ID # | | Mean ± SD | | Mean ± SD | | Mean ± SD | | Mean ± SD |
| Ambient | 23.2-24.5 | S1 | 25.0 | 18.4 ± 8.9 | 23.4 | 35.5 ± 14.2 | 546 | 506 ± 80 | 48.1 | 54.2 ± 13.0 |
| | | S2 | 8.2 | | 51.1 | | 458 | | 69.1 | |
| | | S3 | 22.0 | | 31.9 | | 514 | | 45.3 | |
| Fridge | 6.0-6.3 | S4 | 4.5 | 3.6 ± 1.4 | 15.1 | | 336 | | 24.9 | 25.4 ± 1.2 |
| | | S5 | 2.0 | | 17.6 | 16.5 ± 1.3 | 327 | 319 ± 44 | 26.7 | |
| | | S6 | 4.2 | | 16.9 | | 295 | | 24.5 | |

TABLE 8.2
BMP-7 extracted from human NMP processed at 18° C.

| Test | | AcOH | TBS | AcOH | TBS | AcOH | TBS |
|---|---|---|---|---|---|---|---|
| Article | | BMP-7 | BMP-7 | TGF-β1 | TGF-β1 | IGF-2 | IGF-2 |
| ID | Form | (ng/g) | (ng/g) | (ng/g) | (ng/g) | (ng/g) | (ng/g) |
| 8-1 | Microparticulate | 10.99 | 19.65 | 23.75 | 1.70 | >ULQ | >ULQ |
| 8-2 | Microparticulate | 9.55 | 14.93 | 9.34 | 1.24 | >ULQ | >ULQ |
| 8-3 | Microparticulate | 6.25 | 15.84 | 15.27 | 1.05 | >ULQ | >ULQ |
| 8-4 | Microparticulate | 3.08 | 6.10 | 10.69 | 0.60 | >ULQ | >ULQ |
| 8-5 | Fibers | 0.66 | 12.73 | 1.11 | 1.35 | 67.95 | 65.57 |
| 8-6 | Fibers | 0.57 | 12.90 | 5.48 | 0.96 | 24.44 | >ULQ |
| 8-7 | Fibers | 2.55 | 2.99 | 2.61 | 0.69 | 77.43 | 61.02 |
| 8-8 | Fibers | 0.99 | 5.99 | 6.22 | 0.61 | 65.34 | 77.74 |

“>ULQ” = above upper limit of quantitation (for IGF-2 this was >80 ng/g)

### Discussion

From study 8.1 it can be seen that the amount of BMP-7 released into AcOH and TBS was approximately 2 and 5 fold higher respectively in the NMP prepared at ambient temperature compared to the NMP prepared in the fridge. Similar results were observed for TGF-ß1.

From study 8.2 it can be seen that when human NMP microparticulate (0.25 to 1 mm) was processed at 18±1° C. the AcOH extractable BMP-7 ranged from 3.08 to 10.99 ng/g while the fibers ranged from 0.57 to 2.55ng/g in the AcOH extract. In comparison the human DBM microparticulate (0.25 to 1 mm) and DBM fibers produced in Example 6 had undetectable and 0.26ng/g of AcOH extractable BMP-7 indicating that the NMP process is effective at 18° C. We also observed that all the NMP fibers and NMP microparticulate samples had measurable levels of TGF-ß1 (1.11 to 23.75ng/g) and IGF-2 (24.4 to >80ng/g) in the AcOH extracts. The TBS extracts had higher levels of BMP-7 than the AcOH extracts for all test articles, while the AcOH extracts had higher levels of TGF-ß1 than the TBS extracts. The AcOH and TBS extracts had similar levels of IGF-2.

### Example 9 In Vivo Osteoinductive Activity Testing of NMP

Sterile NMP samples with AcOH extractable BMP-7 levels of 0.72 (S9-1), 1.16 (S9-2) and 3.37 (S9-3) ng/g were tested for osteoinductive activity in an athymic rat muscle pouch model and the amount of bone formed was assessed by microCT and histology according to ASTM 2529-13 and Barr et al. Oral Surg Oral Med Oral Pathol Oral Radiol Endod (2010) April; 109(4):531-40.

NMP samples were hydrated with sterile saline and loaded into 1cc syringes and a fixed volume of graft was placed in the muscle pouch formed in both hind limbs of the rat. NMP S9-1 had 10 implants placed while S9-2 and S9-3 had 5 implants each placed.

After 28 days the rats were euthanized and the implants recovered and placed in 10% formalin. They were then scanned by microCT (Quantum FX, Perkin Elmer, MA), demineralized, embedded in wax, section and stained with Hematoxylin and Eosin.

The mineral content of the microCT scans was calculated by comparison to a phantom that was scanned at the same time and the explanted tissue was segmented into bone and non-bone using 200 mg HA/cc as the threshold.

### Results

The bone volume by microCT results are summarized in Table 9.1. MicroCT analysis showed that the group with the highest AcOH extractable BMP-7 produced the most bone.

Histologically all groups had woven bone forming within the body of the implant.

TABLE 9.1
Summary of microCT results

| | AcOH BMP-7 | Bone Volume |
|---|---|---|
| Group | (ng/mL) | (mm3) |
| S9-1 | 0.72 | 9.36 ± 5.32 |
| S9-2 | 1.16 | 14.95 ± 9.96 |
| S9-3 | 3.37 | 50.10 ± 29.05 |

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to limit the invention in any way. The disclosures of all prior art cited herein are incorporated by reference in their entirety.

1. A process for treating demineralized bone (DB) comprising:
incubating the DB with an aqueous solution of a chaotropic agent at a temperature in the range of about 12° C. to about 30° C. to increase the release of a bone growth factor; and
selectively removing the chaotropic agent while retaining the released growth factor and the DB.

2. A process for treating demineralized bone (DB) comprising:
incubating the DB with an aqueous solution of a chaotropic agent at a temperature in the range of about 2° C. to about 30° C. to increase the release of a bone growth factor; and
selectively removing the chaotropic agent while retaining the released growth factor and DB, and
wherein the process is carried out in the absence of added protease inhibitor.

3. The process of claim 1 wherein the solution of chaotropic agent further includes a salt.

4. The process of claim 1 wherein the selective removal of chaotropic agents comprises filtration through a semi-permeable membrane.

5. The process of claim 4 wherein the semi-permeable membrane has a molecular weight cut off (MWCO) of between about 1 and about 25 KDa.

6. (canceled)

7. The process of claim 1 wherein the chaotropic agent is urea or guanidine HCl.

8. The process of claim 3 wherein the salt is calcium chloride, sodium chloride, or potassium chloride.

9. (canceled)

10. The process of claim 1 wherein the chaotropic agent is urea and the urea concentration is in the range of about 4M to about 8M.

11. The process of claim 3 wherein the salt is calcium chloride, and the calcium chloride concentration is between 0.25 and 0.75M.

12-13. (canceled)

14. The process of claim 1 wherein the incubation is for at least 4 hours.

15. The process of claim 1 wherein the incubation is conducted at a pH in the range of 5 to 8.

16. (canceled)

17. The process of claim 1 wherein the bone growth factor is one or more of a bone morphogenic protein (BMP), TGF-ß1, PDGF-BB, VEGF and IGF-2.

18-19. (canceled)

20. The process of claim 1 wherein the selective removal of the chaotropic agent is carried out by dialysis.

21. (canceled)

22. The process of claim 1 wherein the process further comprises concentrating the demineralized bone and the growth factor by removing water.

23. The process of claim 22 wherein following the concentrating, demineralized bone and growth factor are lyophilized.

24. The process of claim 1 wherein the process is carried out in the absence of a protease inhibitor.

25. A product prepared by the process of claim 1.

26-27. (canceled)

28. A method of promoting bone formation or bone fusion comprising administering to a patient in need thereof the product of claim 25.

29. The method of claim 28 wherein promoting bone formation or bone fusion comprises administering the product into boney voids or bone defects, or at the site where fusion of non-unions, or fusion of two bones is to occur.

30-31. (canceled)

32. A process for treating bone matrix comprising:
placing bone matrix in a dialysis bag;
demineralizing the bone matrix and incubating the bone matrix with a chaotropic agent at a temperature in the range of about 12° C. to about 30° C. to increase the release of a bone growth factor;
selectively removing the chaotropic agent while retaining the released growth factor; and
the demineralized bone (DB).
